(19) United States
(12) Patent Application Publication
(10) Pub. No.: US 2011/0070202 A1
(43) Pub. Date: Mar. 24, 2011

(54) Targeted delivery to the brain of magnetically labeled stem cells

(76) Inventors: Paul Yarowsky, Bethesda, MD (US); Paul Fishman, Baltimore, MD (US); Adam Puche, Ellicottt City, MD (US); Wei-Ben Shen, Ellicott City, MD (US)

(21) Appl. No.: 12/807,999

(22) Filed: Sep. 17, 2010

Related U.S. Application Data
(60) Provisional application No. 61/243,737, filed on Sep. 18, 2009.

(51) Int. Cl. A61K 35/12; A61P 25/00

(52) U.S. Cl. 424/93.7

(57) ABSTRACT

Provided herein is a method of targeting stem cells to a specific region of the central nervous system of an individual in need of such treatment, comprising the steps of administering stem cells containing a superparamagnetic nanoparticles to said individual; and applying an external source of a magnetic field to the exterior of said individual's brain so as to advance and maneuver said stem cells. Also provided is a composition for targeting cells to a specific region of the central nervous system of an individual in need of such treatment, comprising stem cells containing a superparamagnetic nanoparticles.

### CROSS REFERENCE TO RELATED APPLICATION

This non-provisional application claims the benefit of priority under 35 U.S.C. §119(e) of provisional U.S. Ser. No. 61/243,737, filed Sep. 18, 2009, now abandoned, the entirety of which is hereby incorporated by reference.

### BACKGROUND OF THE INVENTION

1. Field of the invention

The present invention relates to the fields of neurology and stem cell therapeutics. More specifically, the present invention relates to methods for targeted delivery to the brain of magnetically labeled stem cells.

2. Description of the Related Art

The ability to apply forces on magnetic nanoparticles with external magnetic fields has been harnessed in biomedical applications including prosthetics (1), drug delivery (2) and antiangiogenesis strategies (3-4). U.S. Pat. No. 4,247,406 describes an intravascularly-administrable, magnetically-localizable biodegradable carrier comprising microspheres formed from an amino acid polymer matrix containing magnetic nanoparticles embedded within the matrix for targeted delivery of chemotherapeutic agents to cancer patients. Microspheres with magnetic nanoparticles, which are suggested to enhance binding of a carrier to the receptors of capillary endothelial cells when under the influence of a suitable magnetic field, are also described in U.S. Pat. No. 5,129,877.

U.S. Pat. Nos. 6,375,606; 6,315,709; 6,296,604; and 6,364,823 describe methods and compositions for treating vascular defects with a mixture of biocompatible polymer material, a biocompatible solvent, adhesive and preferably magnetic nanoparticles to control delivery of the mixture. In these methods, a magnetic coil or ferrofluid is delivered via catheter into the aneurysm. This magnetic device is shaped, delivered, steered and held in place using external magnetic fields and/or gradients.

A model for inducing highly localized phase transformations at defined locations in the vascular system by applying 1) external uniform magnetic fields to an injected superparamagnetic colloidal fluid for the purpose of magnetization and 2) using embedded particles to create high magnetic field gradients was described (5). This work describes the use of uniform magnetic fields in combination with magnetic particles (greater than 2 micron in diameter) to form chains along the direction of applied field and in turn use this to embolize micro-vessels (50-100 microns in diameter).

Known methods and devices for delivery of magnetizable drug or agent-containing magnetic carrier to specific locations in the body rely upon a single source of magnetic field to both magnetize the carriers and to pull them by magnetic force to the specific location. Previous attempts to use magnetic particles in these applications have relied on high gradient magnetic fields produced by magnets external to the body to direct magnetic particles to specific locations. The main disadvantage of this approach is that externally generated magnetic fields apply relatively small and insufficiently local forces on micron and nano-scale magnetic particles, and thus these methods have limited applications.

Gordon in U.S. Patent Publication No. US 2002/0133225 describes a device comprising an implant having a magnetic field and a medical agent carried by a magnetically sensitive carrier. The carrier is introduced into the blood flow of the organism upstream from the target tissue, and the carrier and medical agent migrate via the blood flow to the target tissue. Gordon discloses an implant comprising a magnetized material (e.g., a ferromagnetic or a superparamagnetic material). Examples describe making a stent from ferromagnetic materials and magnetized by using an external magnet or made from a magnetized material.

Traumatic brain injury (TBI) has been a major source of morbidity and mortality in military conflicts, but with the more recent introduction of improvised anti-personnel devices (IADs) and associated blast injuries, traumatic brain injury has become the leading cause of disability to veterans of the armed forces. Along with its immediate and long lasting consequences, there is increasing evidence that traumatic brain injury is associated with later development of dementia and Alzheimer's disease (AD) (6).

One of the most promising novel forms of therapy for both acute and chronic traumatic brain injury is the use of stem cells either neural or endothelial progenitor cells. These cell-based therapies can promote in animal models blockade of programmed cell death, both caspase-dependent and caspase-independent, neurogenesis and angiogenesis in the lesion, with restoration of neurological function after brain injuries (7-9). This suggests that stem cell transplantation is a potential therapy for brain injury. However, fundamental issues such as the delivery of these cells to regions of injury within the brain as well as their survival remain to be resolved.

Methods for delivering stem cells in traumatic brain injury with maximum efficiency and specificity and at the same time with minimal risks have gained only limited attention. The need for specificity depends on the ability for homing, diapedesis, and migration of the transplanted cells. The chemoattractive gradient released at the site of traumatic brain injury leads to the homing of neural progenitor cells in the brain lesion similar to that seen in focal ischemia (10).

A number of studies have revealed limitations of acute intra-arterial, intracisternal and intravenous stem cell therapy (10). These techniques allow only a few percent of the stem cells injected to reach the sites of trauma. In the case of intravenous injection, the lung and the spleen act as traps for the cells before they reach the brain's vessels (9-10). Even direct intracerebral stem cell injections following traumatic brain injury, yield only 1.4-1.9% of the infused cells at 48 h post injection (11).

There are a number of reasons for the low yield of stem cells at the site of lesion using these methods. With intravenous injection, most of the cells are trapped in the lungs and the spleen before reaching the brain (9). For intracisternal delivery, most of the cells are washed out before transependymal movement allows for diapedesis. For intra-arterial injection, arterial blood flow rates are high resulting in washing the cells through the lesion prior to extravasation. In addition, there is a low yield of surviving stem cells because there is increased caspase activation at the lesion. This increase caspase activation has been described in both human traumatic brain injury and in animal models of traumatic brain injury (12).

Thus, there is a recognized need in the art for effective therapeutic methods of regulating the intrinsic capability of stem cells to migrate within the brain which is an essential requirement for stem cell transplantation therapy. The present invention fulfils this longstanding need in the art.

### SUMMARY OF THE INVENTION

The present invention describes the use of external magnets to direct genetically engineered labeled stem cells to the site of cortical lesions in traumatic brain injury, based upon an applied magnetic field. Once localized to the site of injury further migratory ability of the cells into the damaged region is necessary. Metalloproteinases (MMPs) are involved in cell migration both within the brain (across the extracellular matrix) and across the extracellular matrix and endothelial cells (Blood Brain Barrier (BBB)). The migratory capability of stem cells may be enhanced by transient transfection of the membrane type 5 matrix metalloproteinase (MT5-MMP). Metalloproteinases are implicated in cell migration and play a role in migration of endogenous neural stem cells. Currently, there is no technology available to improve the targeting, retention, and migration of cells injected into the nervous system.

There are a number of reasons for the current low yield of stem cells at the site of brain lesions using intravenous, intra-cisternal, or intra-arterial injections. With intravenous injection, most of the cells are trapped in the lungs and the spleen before reaching the brain (9,13). For intracisternal delivery, most of the cells are washed out before transependymal movement allows for their diapedesis into the parenchyma. For intra-arterial injection, blood flow rates are so high that the size of the lesion field penetrated by stem cells is unpredictable. The present invention discloses efficient stem cell delivery in traumatic brain injury by developing an approach that uses both a magnetic field to target specific cortical regions and a genetic approach that uses transient overexpression of a specific metalloproteinase, MT5-MMP.

Thus, the present invention is directed to a method of targeting stem cells to a specific region of the central nervous system of an individual in need of such treatment, comprising the steps of administering stem cells containing superparamagnetic nanoparticles to said individual; and applying an external source of a magnetic field to the exterior of said individual's brain so as to advance and maneuver said stem cells, thus targeting the stem cells to a specific region of the central nervous system of the individual.

The present invention also is directed to a composition for targeting cells to a specific region of the central nervous system of an individual in need of such treatment, comprising stem cells containing superparamagnetic nanoparticles.

The present invention is directed further to a method of targeting superparamagnetic nanoparticles containing human neuroprogenitor stem cells to a specific region of the central nervous system of an individual in need of such treatment, comprising the steps of administering said stem cells to said individual; and applying an external source of a magnetic field to the exterior of said individual's brain so as to advance and maneuver said stem cells.

Other and further aspects, features, benefits, and advantages of the present invention will be apparent from the following description of the presently preferred embodiments of the invention given for the purpose of disclosure.

### BRIEF DESCRIPTION OF THE DRAWINGS

FIGS. 1A-1H show the effects of neodymium magnet on migration of D3 stem cells in vitro. To investigate the effects of a magnetic field on migration in vitro, a neodymium magnet (diameter=¼″, thickness=⅛-¼″, 0.3-0.6T) was placed below the center of a culture dish (diameter, 150 mm) containing Feridex-labeled D3 cells in Matrigel-coated dishes (FIG. 1G). D3 cells migrated toward the magnet depending upon the strength of the magnet over 48 hr (FIG. 1H). Such migration was not seen in control (non-Feridex-labeled D3) dishes. FIG. 1A, FIG. 1B, and FIG. 1C are confocal images of Feridex-labeled D3 cells stained with the nuclear dye DAPI. FIG. 1D, FIG. 1E, and FIG. 1F are high-power images (×40) of a representative field in FIG. 1A, FIG. 1B, and FIG. 1C.

FIGS. 2A-2C show the effects of neodymium magnet on migration of neuroprogenitor stem cells in vivo. Intra-ventricular injection of Feridex-labeled neuroprogenitor stem cells. Seven days after injection of neuroprogenitor stem cells, animals were prepared for histology. Dextran antibody labeled Feridex-NPC (green cells). Ipsilateral migration of stem cells occurred throughout the cortex in the animal with a surface magnet (FIG. 2B) but at a significantly lower level in the control (FIG. 2A) animal without a magnet. A count of the numbers of cells labeled/mm² is shown in (FIG. 2C). Red is control and Blue is magnet.

FIGS. 3A-3E show the effects of neodymium magnet on migration of NPC Feridex-labeled cells in TBI. Rats (2) were given CCI. Twenty four hr later, they were given neuroprogenitor stem cells (3×10⁶ cells) by IA injection in the absence of a magnet (FIG. 3A) and in the presence of a 0.6 T neodymium magnet, attached to the outside of the skull with high-strength adhesive prior to the injection of the neuroprogenitor stem cells (FIG. 3B). Two hours later, the animals were euthanized and the brains fixed with 4% paraformaldehyde. Controls (2, non-TBI) were similarly injected with Feridex-labeled neuroprogenitor stem cells in the absence (FIG. 3C) and in the presence (FIG. 3D) of the magnet. Sections (30 um) were stained with Prussian Blue to visualized Feridex. Cell counts were performed only within the vessel identified by the presence of light brown red blood cell accumulation (FIG. 3E).

FIG. 4 shows a magnetic transfer of stem cells in the cortex. The magnet is placed ipsilaterally on the skull and the stem cells are injected into the lateral ventricle (*) or ipsilateral carotid artery. The greatest magnetic field is on the ipsilateral cortex, and the contra-lateral cortex serves as the internal control.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.”

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, the term “superparamagnetic particle”, refers to a material that conducts magnetic flux strongly. Examples of superparamagnetic nanoparticles useful in the magnetizable cells of the present invention include, but are not limited to, cobalt, iron, iron oxides, nickel, and rare earth magnetic materials and various soft magnetic alloys (e.g., Ni—Co). In one embodiment, the superparamagnetic nanoparticles are magnetized only in the presence of externally applied magnetic fields. Examples of these types of magnetizable particles include, but are not limited to, superparamagnets and soft ferromagnets. In other embodiments, magnetizable particles known as ferromagnets, are used.

In one embodiment of the present invention, there is provided a method of targeting stem cells to a specific region of the central nervous system of an individual in need of such treatment, comprising the steps of administering stem cells containing superparamagnetic nanoparticles to the individual; and applying an external source of a magnetic field to the exterior of the individual's brain so as to advance and maneuver the stem cells, thus targeting the stem cells to a specific region of the central nervous system. Although it is contemplated that any stem cells may be targeted using this method, human neuroprogenitor stem cells are particularly preferred. A variety of superparamagnetic nanoparticles may be used in the methods of the present invention with representative particles including, but not limited to, Feridex, (Fe₂O₃), Fe₃O₄, FeNi and FePt. In this method, the stem cells may be administered to the brain in a variety of ways, but representative routes include but are not limited to intraventricular administration, intraparenchymal injection and intra-arterial injection. It is further contemplated that the methods of the present invention may be enhanced by augmenting the ability of the stem cells to traverse to the desired site and survive within the nervous system. For example, the stem cells may contain a growth factor or expression of heat shock protein 70 (Hsp70) may be upregulated in the stem cells. Furthermore, the stem cells may be transfected with a metalloproteinase. A representative example of a metalloproteinase is MT5-MMP. A person having ordinary skill in this art would recognize useful external sources of a magnetic field. In a preferred aspect, the fixed magnet has a strength between 0.3T and 6.0T. The amount of stem cells and the volume in which the stem cells are administered may be manipulated by one having ordinary skill in this art to fit the purpose of the treatment. Generally, the amount of cells administered is from about 40,000 cells/μl-80,000 cells/μl in a volume of from 0.5-2.0 ml. It is contemplated that the methods of the present invention may be used to treat a variety of neurological conditions, including but not limited to traumatic brain injury (TBI), Parkinson's Disease, Alzheimer's Disease, stroke and amyotrophic lateral sclerosis (ALS). As would be well known by a person having ordinary skill in this art, to treat Parkinson's Disease, the cells are targeted to the striatum; for ALS to the spinal cord, and to the cerebral cortex for Alzheimer's Disease.

In another embodiment of the present invention, there is provided a composition for targeting cells to a specific region of the central nervous system of an individual in need of such treatment, comprising stem cells containing superparamagnetic nanoparticles. In a preferred aspect of this embodiment, the stem cells are human neuroprogenitor stem cells. Representative superparamagnetic nanoparticles are described above. The stem cells may contain a growth factor or expression of heat shock protein 70 may be upregulated in the stem cells. Furthermore, the stem cells may be transfected with a metalloproteinase. A representative example of a metalloproteinase is MT5-MMP.

In yet another embodiment of the present invention, there is provided a method of targeting superparamagnetic particle-containing human neuroprogenitor stem cells to a specific region of the central nervous system of an individual in need of such treatment, comprising the steps of administering the stem cells to the individual; and applying an external source of a magnetic field to the exterior of the individual's brain so as to advance and maneuver the stem cells. A variety of superparamagnetic nanoparticles may be used in the methods of the present invention with representative nanoparticles including, but not limited to, Feridex (Fe₂O₃), Fe₃O₄, FeNi, and FePt. In this method, the stem cells may be administered in a variety of ways, but representative routes include but are not limited to intraventricular administration and intra-arterial injection. It is further contemplated that the methods of the present invention may be enhanced by augmenting the ability of the stem cells to traverse to the desired site and survive within the nervous system. For example, the stem cells may contain a growth factor or expression of Hsp70 may be upregulated in the stem cells. Furthermore, the stem cells may be transfected with a metalloproteinase. A representative example of a metalloproteinase is MT5-MMP that can enable stem cells to traverse the Blood Brain Barrier (BBB). A person having ordinary skill in this art would recognize useful external sources of a magnetic field. In a preferred aspect, the fixed magnet has a strength between 0.3T and 6.0T. The amount of stem cells and the volume in which the stem cells are administered may be manipulated by one having ordinary skill in this art to fit the purpose of the treatment. Generally, the amount of cells administered is from about 40,000/μl-80,000 cells/μl in a volume of from 0.5-2.0 ml. It is contemplated that the methods of the present invention may be used to treat a variety of neurological conditions, including but not limited to traumatic brain injury, Parkinson's Disease, Alzheimer's Disease, Amyotrophic Lateral Sclerosis and stroke.

The following examples are given for the purpose of illustrating various embodiments of the invention and are not meant to limit the present invention in any fashion.

### EXAMPLES

Application of a magnetic field and metalloproteinase enhanced migration will improve stem cell targeting, retention, and migration at the cortical lesion in TBI

Targeting of Feridex-labeled human neuroprogenitor stem cells (NPCs) to damaged cortical regions following traumatic brain injury when introduced by different routes of administration is assessed quantitatively. One route of administration is intraventricular (IVt) administration. Intraventricular delivery is advantageous by distributing the cells widely below the region of cortical traumatic brain injury. The present invention discloses that a surface-applied magnetic field will attract the Feridex labeled stem cells to the ventricular surface of the cortex and encourage transependymal movement into the cortex. Metalloproteinase transfection should enhance the ability of the cells to migrate up into the cortex into the cortical lesion and its penumbra.

Another route of administration is intra-arterial (IA) injection. Intra-arterial delivery is the least invasive as it does not involve injection into brain tissues. An external magnetic field with Feridex labeled stem cells will enhance localization within the capillary plexus of the brain in the region of the lesion, i.e. the traumatic brain injury site. Enhancing metalloproteinases will enhance the extravasation ability of the cells similar to the metalloproteinase enhanced metastasis of cancer stem cells. Currently there is no technology available to improve the targeting, retention, and migration of cells injected into the nervous system.

### Models of TBI and Use of NPCs

One model of experimental traumatic brain injury that has been used is the controlled cortical impact (CCI) model. This model is used for generating focal contusions and has been shown to be histopathologically similar to patients with penetrating injury traumatic brain injury. A second system of experimental traumatic brain injury is known as lateral fluid percussion injury model (LFPI). For this technique, brain injury in a rat is induced by rapidly injecting saline into the closed skull. The third model is the diffuse traumatic brain injury model in the rat (14).

Traumatic brain injury by controlled cortical impact of the left parietal cortex (7,11,15) is used herein. This model induces both necrotic and apoptotic cell death, causing brain lesions and the development of behavioral deficits. The controlled cortical impact model is used in both mice and rats and causes severe localized injury where the effectiveness of a stem cell therapeutic intervention can be analyzed. Also, the controlled cortical impact model transiently increases permeability of the BBB (14,16), which in the case of stem cell therapy has great advantages for stem cell delivery to the site of the lesion. Transplantation of neural progenitor cells following experimental traumatic brain injury has been used to improve functional recovery (7,11). Therapeutic benefits gained from cell-based therapy depend on migration and localization of grafted cells within the target tissue (17).

### Feridex-Labeling of Stem Cells

The present invention discloses the use of stem cells labeled with iron oxide nanoparticles coupled with external magnets to direct the location of stem cell distribution by either intraventricular or intra-arterial injection. Feridex nanoparticles are an FDA approved MRI contrast agent consisting of an iron particle with a dextran coat. Five different lines stem cell lines can be non-destructively loaded with Feridex at a loading efficiency of ˜90% of cells with cell death less than 5%. These cells were attracted to an external magnet in vitro and in vivo in the CNS as described below.

Previously, cells labeled with iron oxide nanoparticles have been utilized to monitor the cell migration on MRI in various experimental disease models of neurodegenerative diseases in limited areas. Iron oxide (Feridex)-labeled neural progenitor cells have similar use in neuronal differentiation and axon growth compared with nonlabeled neural progenitor cells, and thus there are few toxic effects of Feridex labeling neural progenitor cells.

The theory behind this method is that superparamagnetic iron oxide (SPIO) nanoparticles, such as Feridex, are incorporated into endosomes of stem cells and thus are not considered free intracellular iron which could cause cellular damage. This method works for a variety of nonphagocytic, nondividing or phagocytic rapidly dividing cells. The magnetic labeling technique has been shown to have no short- or long-term toxic effects to cells at the site of transplantation. The incorporation of Feridex into the endosomes results in the cell itself acting as a magnetic resonance superparamagnetic contrast agent.

In addition, Feridex-labeled cells are attracted to magnets and therefore can be moved by a magnetic field gradient. By labeling neural progenitor cells with Feridex, these cells can then be separated from unlabeled cells using magnetic columns (magnetic activated cell sorter) that are used for cell sorting using antibody-labeled magnetic beads.

Therefore, the present invention discloses that with the application of a magnetic field, Feridex-labeled stem cells can be targeted or directed to certain regions of the CNS after an injection, thus facilitating the retention of these labeled cells in the targeted tissue for extended periods of time. By directing these Feridex-labeled cells to areas of tissue damage, one can deliver stem cells that stimulate maintaining tissue homeostasis by supplying missing growth factors and slowing down further neurodegeneration.

Magnetic cell targeting to facilitate stem cell migration has had very limited investigation to date. External magnets had been shown to enhance the retention of intravenously injected stem cells to the liver. Similar enhancement of stem cell delivery from the blood has been shown in a rat model of myocardial infarction. The only published experience with this method in the brain or spinal cord is very recent work in a focal ischemia model, which has shown that that intravenous injection of a human neural stem cell line increased by three fold the iron content of the cortex on the same side as the stroke and decreased the infarct size by 20%. The disadvantages with this technique is that intravenous injection allows only a small number of cells to arrive in the target area, since the majority of the cells are trapped in the lung and spleen (9). In addition, there is a low survival rate of grafted cells in ischemia (7,11). No application for the use of this method in the brain or spinal cord following traumatic injury has been attempted.

### Matrix Metalloproteinases

Individual stem cells migrating in a mature brain milieu have to ‘push’ through an established extracellular matrix environment. Recent studies on the migration of mouse stem cells in brain suggest a role for the matrix metalloproteinases in this process. Metalloproteinases are zinc-dependent proteolytic enzymes involved in the remodeling of extracellular matrix. At present, there are more than 25 metalloproteinase members and, collectively, the family of metalloproteinases can cleave all components of the extracellular matrix as well as allow cells to pass through endothelial cells. Metalloproteinase activity is highly regulated at both the transcriptional and translational level, and by the presence of tissue inhibitors of metalloproteinases. Metalloproteinases mediate cell migration by breakdown of extracellular matrix barriers which impede cell movement. Little is known of the role of metalloproteinases in neuronal migration.

Some members of the metalloproteinases, specifically MT5-MMP, may play a role in the migration of mouse neuronal stem cells in the olfactory bulb. It has also been shown that inhibition of MT5-MMP in vitro retards stem cell migration. Thus engineering of human stem cells to transiently express MT5-MMP, will allow injected neural stem cells reaching the target's vascular bed to enter the parenchyma through adhesion and diapedesis paralleling the mechanism used by inflammatory cells. Furthermore, metalloproteinases are highly associated with metastatic ability in cancer stem cells. Thus, the present invention discloses that overexpression of the target metalloproteinase enhances the ability of stem cells to exit the blood vessels and enter the brain parenchyma in an intra-arterial delivery model. At the molecular level, matrix metalloproteinases and the SDF-1/CXCR4 system are involved in directed cell migration in the brain.

Use of NPCs

Transplantation of neural progenitor cells (NPCs) following experimental traumatic brain injury has been used to improve functional recovery (17). Therapeutic benefits gained from cell-based therapy depend on migration and localization of grafted cells within the target tissue.

Preliminary Studies

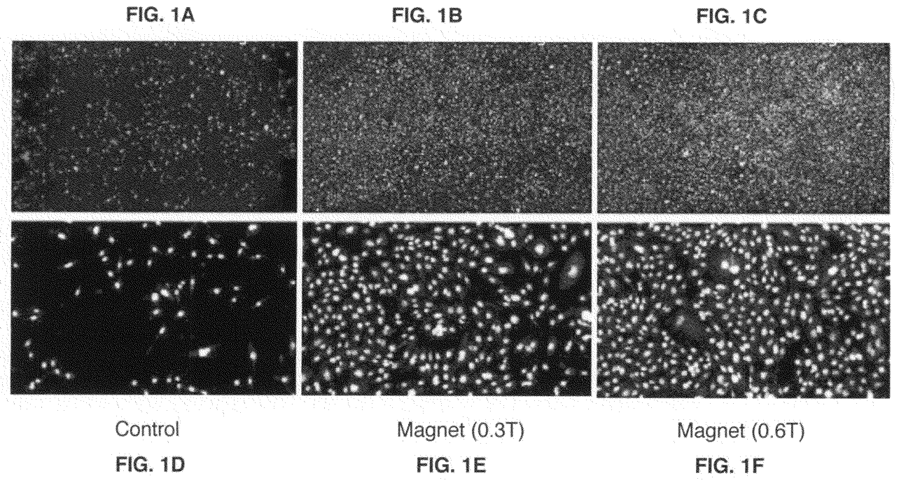

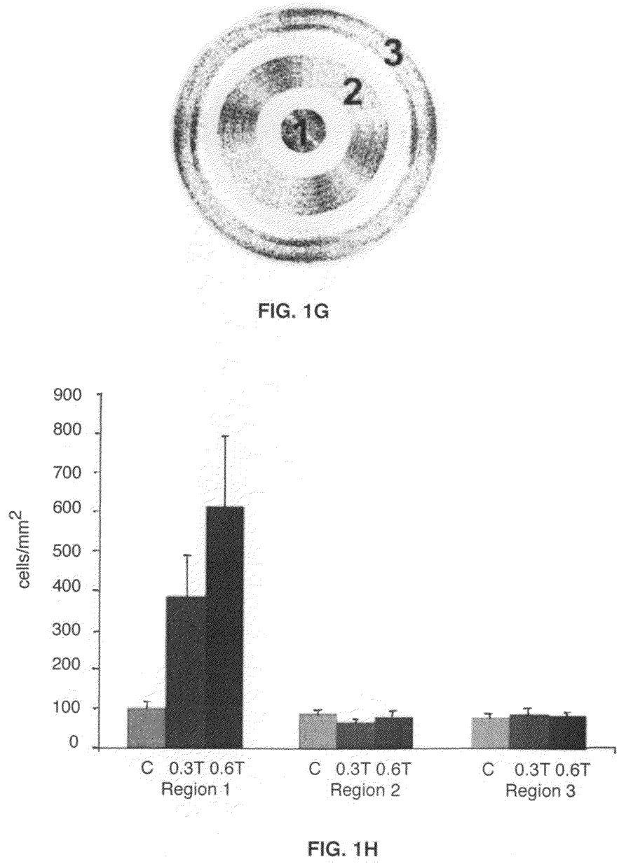

The properties of Feridex-labeled human neural progenitor cells are investigated. In preliminary experiments, the in vitro migration of Feridex-labeled D3 mouse embryonic stem cells was studied in a magnetic field. The confocal images in FIGS. 1B-1C and the high-power images in FIGS. 1E-1F of DAPI-stained Feridex-labeled D3 cells depict the migration of the cells toward the magnet compared to control non-labeled D3 cells in FIGS. 1A, 1D. The numbers of cells that moved toward the magnet increased as the magnetic field strength increased (FIGS. 1G-1H).

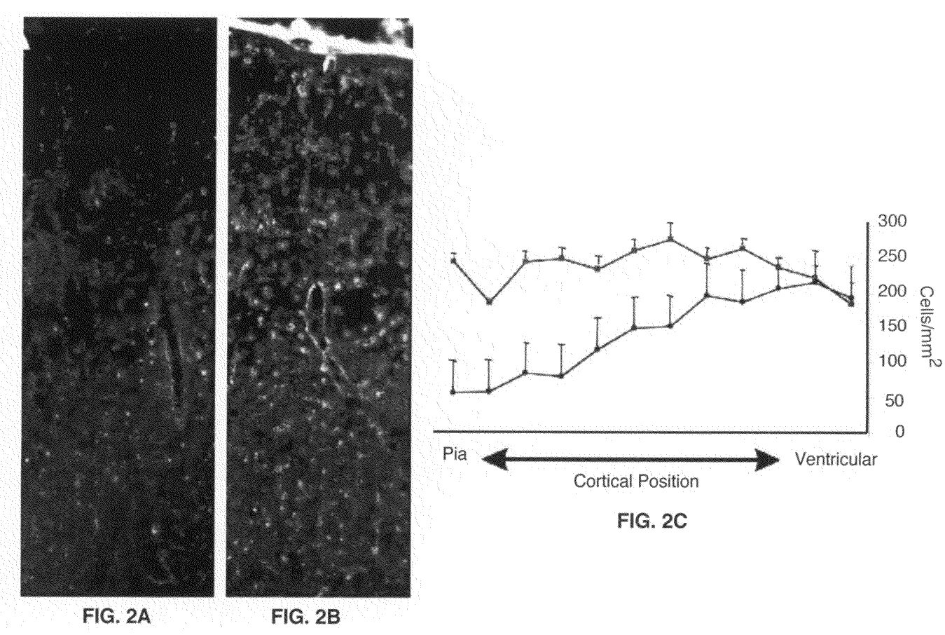

In an in vivo study, Feridex-labeled neural progenitor cells were delivered into the ventricles (lateral) in normal rats to examine the capacity of the magnetic field on migration of these cells into the brain. Ninety day old rats were anesthetized and Feridex-labeled neural progenitor cells injected into one of the lateral ventricles. A 0.6 T fixed magnet was placed ipsilateral to the injection of neural progenitor cells. A large number of Feridex-labeled neural progenitor cells migrated all the way to the marginal zone (MZ) of the cerebral cortex on the side that the magnet is located after 7 days (FIGS. 2B-2C). In contrast, control animals lacking a magnet exhibited the majority of cells only entering the ventral most layer 5/6 of the cortex and significantly fewer cells reaching the apical regions (FIGS. 2A, 2C).

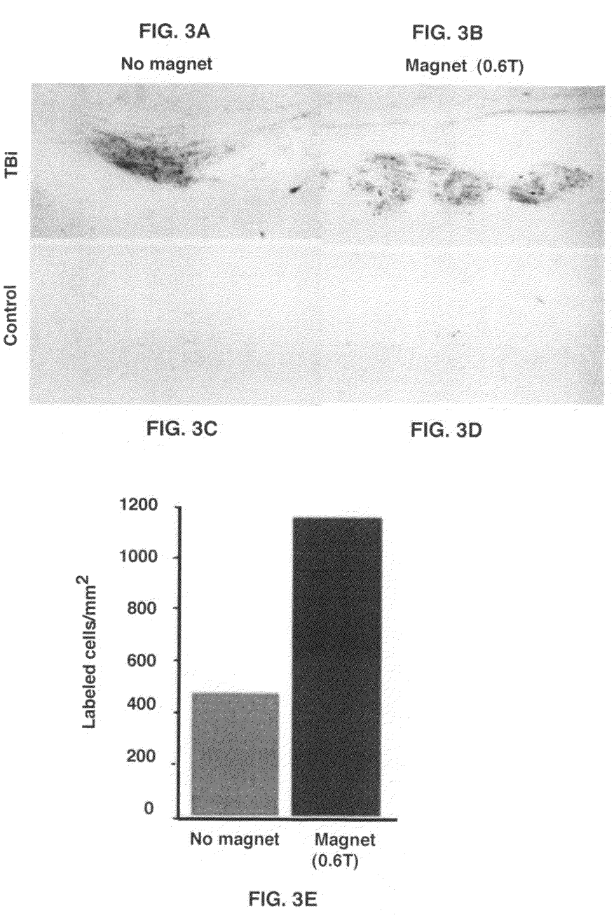

In a second study, traumatic brain injury was induced in rats via the cortical impact model and 24 hours later Feridex labeled neuroprogenitor stem cells were delivered by intra-arterial injection. Samples of blood were removed from the tail vein at 15 minute intervals and the number of circulating stem cells assessed. Within 30 minutes there were no Feridex labeled stem cells in circulation. The brains were removed 2 hours following stem cell delivery and processed for histology. In control animals that did not receive traumatic brain injury, only rare scattered Feridex labeled stem cells were found in brains upon which a 0.6T fixed magnet was placed over the cortex (FIGS. 3C-3D). However, in traumatic brain injury animals, large vessels below the region of traumatic brain injury contained numerous Feridex labeled neural progenitor cells. In the animal containing a 0.6T fixed magnet atop the skull above the region of traumatic brain injury (FIG. 3B), the density of Feridex labeled stem cells within this large vessel was 2.4 times greater (FIG. 3E) than the density present in the no-magnet traumatic brain injury brain (FIG. 3A).

These experiments suggest that this magnetic field enhanced migratory strategy is highly effective at improving the distances that stem cells will traverse following intraventricular delivery and increasing the retention of stem cells in a traumatic brain injury region following intra-arterial delivery. Thus, this approach will enhance stem cell migration following traumatic brain injury. As a control on the fate of Feridex-labeled stem cells, whether superparamagnetic iron oxide nanoparticle get out of neural progenitor cells and are taken up by microglia was investigated. No huge increase in the numbers of microglia labeled with Feridex was found.

### Procedure

In experiments, traumatic brain injury is performed by controlled cortical impact of the left parietal cortex. Four groups of rats are used to evaluate targeted stem cell migration for each delivery method. For each group, either an intraventricular injection or an intra-arterial injection is performed one day after the animal receives traumatic brain injury. Before delivery and Feridex-labeling (for 24 hr), neural progenitor cells are either transiently transfected with a bicistronic construct consisting of the metalloproteinase gene MT5-MMP and GFP (available from Genecopia) or transfected only with GFP. Approximately 35% transfection efficiency has been achieved with these constructs. These transient expressions were monitored and elevated proteins are present for 2 weeks following transfection. Non-transfected cells serve as an internal control, but as transfected cells may indirectly affect the non-transfected cells, separate control groups of animals are also needed.

Evaluation of the targeting, migration, and survival of the stem cells in traumatic brain injury animals is at 2 hours (targeting), 1 day (migration), and 7 days (migration and survival) after the injection. This time span is sufficient to assess if magnetic fields have enhanced retention and migration at the region of traumatic brain injury. In addition, experiments are also run on a small number of control animals without traumatic brain injury, using intraventricular and intra-arterial injection of neural progenitor cells. The initial observation was confirmed and expanded that in the presence of traumatic brain injury, there is an increase in stem cell targeting and migration at 2 h, 1 d, and 7 d after the injection as compared to non-traumatic brain injury animals (2 h after injection, see FIG. 3B).

For each type of injection, the four groups consist of: (1) neural progenitor cells, Feridex labeled, are injected into traumatic brain injury animals, one d after the injury with a magnetic field applied during and after injection. (2) neural progenitor cells, Feridex labeled, are injected into traumatic brain injury animals one d after the injury. (magnet and MT5-MMP control). (3) neural progenitor cells are first transfected with MT5-MMP-GFP for 4 hr, followed by Feridex labeling. These transfected neural progenitor cells are injected in traumatic brain injury animals, one d after the injury with a magnetic field applied during and after injection. (4) neural progenitor cells are first transfected MT5-MMP-GFP for 4 hr, followed by, Feridex labeling. These transfected neural progenitor cells are injected in traumatic brain injury animals, one d after the injury (magnet control).

These experiments show that establishing a local magnetic field as well as transfection of neural progenitor cells with MT5-MMP enhances stem cell targeting/migration in the cortex following traumatic brain injury. Following 2 h, 1 d, and 7 d after the injection, animals are euthanized and brains are immersion-fixed in 4% paraformaldehyde and sectioned for histology. The region of traumatic brain injury is assessed by Nissl stain. The position of all transplanted stem cells is identified by immunohistochemistry for the human nuclear antigen and by Prussian Blue histochemistry (for the Feridex iron nanoparticles). Transfected cells identified by immunohistochemistry or direct confocal visualization of GFP, and the distribution is plotted in each section as in published approaches for comparing the distribution of cells in a 3D environment (18-19). The percentage of transfected cells (identified by immunohistochemistry or direct confocal visualization of GFP) are plotted in each section as in published approaches for comparing the distribution of cells in a 3D environment. Sections are visualized at low magnification and the outline of the cortex contour and region of traumatic brain injury mapped using the Neurolucida computer aided camera lucida system. Regions for cell position analysis are selected by systematic random stereological sampling both within the region of traumatic brain injury and adjacent. The plotted position of the cell relative to the pia/ventricular surfaces are used to calculate and compare straight-line distances from the ventricular injection or as a density of cells in the cortex. The estimated number of cells in the regions, the apical-basal position, and estimated efficiency compared to the total number of cells injected are compared across the groups (multifactorial analysis of variance statistics). Also, by using this multiple identification approach for transplanted neural progenitor cells, phagocytic cells that have taken up Feridex following the death of the neural progenitor cell are not counted as they will not be co-labeled with GFP.

The present invention shows that there are statistically more neural progenitor cells targeted to cortical lesions in traumatic brain injury than seen in the cortex in non-traumatic brain injury animals by either injection method. For intra-arterial injection, blood flow in control rats is so high, that it precludes an effect of the magnet. On the other hand, there is a reduction in blood flow (12) in and near the lesion, so that retention by the magnet of neural progenitor cells is now feasible. The time course of neural progenitor cells appearance in different locations associated with the lesion. At 2 hours after injection, neural progenitor cells are in blood vessels near the lesion site (as seen in FIG. 3B); 24 hours after traumatic brain injury, neural progenitor cells have extravasated into tissue around the lesion; 7 days after traumatic brain injury, neural progenitor cells are scattered on the border between normal and damaged tissue and in the lesion core.

With intraventricular injection, at 2 hours after injection, neural progenitor cells are in the ventricle but up against the ventricular surface due to the magnet ‘pull’; at 24 hours after transplantation, neural progenitor cells are at the ventricular-ependymal wall; at 7 days neural progenitor cells are distributed similar to the distribution found in FIGS. 2A-2C. This position of neural progenitor cells after intraventricular administration is different than with intra-arterial administration.

Thus, intra-arterial will have significant extravasation movement at 24 h but may be impacted by blood flow reducing the initial number of cells localizing to the traumatic brain injury, while intraventricular may have less transependymal movement than intra-arterial but will cover a larger area with higher cell density as CSF flow is very slow. Thus, intraventricular delivery is advantageous by distributing the cells widely below the region of cortical traumatic brain injury. The surface applied magnetic field attracts the Feridex labeled stem cells to the ventricular surface of the cortex and encourages migration high into the cortex. Metalloproteinase transfection enhances the ability of the cells to migrate up into the cortex. Intra-arterial delivery is the least invasive as it does not involve injection into brain tissues. An external magnetic field with Feridex labeled stem cells enhances localization within the capillary plexus of the brain in the region of the field, i.e. the traumatic brain injury site. Enhancing metalloproteinases will enhance the extravasation ability of the cells similar to the metalloproteinase enhanced metastasis of cancer stem cells. Lesion volume affects the grafting process but no proportionality has been found in other studies in the volume of the lesion and the numbers of cells recruited (10). A more reproducible relationship is likely, as the factors influencing migration will not only be the pathological changes at the lesion, but will also be based on the magnetic field applied to the cells.

The time course of targeting and migration is generally that two hours after the transplantation of Feridex-labeled neural progenitor cells, cells are localized following intra-arterial administration, in cortical blood vessels that are damaged in traumatic brain injury. Following intraventricular administration, cells are localized only to the injected lateral ventricle at the boundary of the ventricle and the cortex at two hours. One day after intra-arterial transplantation, neural progenitor cells with MT5-MMP are localized in the cortex near the cortical lesion following transependymal movement. Following intraventricular administration with MT5-MMP cells, neural progenitor cells are in the lower layers of the cortex (5/6). By seven days after intra-arterial transplantation, neural progenitor cells with MT5-MMP are in both the injury penumbra as well as in the post-traumatic lesion core, where bioenergetic status is reduced. Following seven days after intraventricular transplantation, neural progenitor cells with MT5-MMP are in the injury penumbra and just entering the post-traumatic lesion core.

Adjusting the amount of cells injected (increasing) or the volume injected (increasing) may improve the results of targeting. The strength of the applied magnetic field can also be increased at 0.3 and 0.6T fields are well below risk levels (human MRI goes upto 7T). By varying these parameters, it may provide increased time to trap the cells in the capillary plexus (IA) or an increased amount of cells to appear at the ventricular/cortical border (IVt). One concern arises with intra-arterial transplantation of stem cells potentially resulting in thromboembolic ischemia. However, in several injections of non-TBI animals no embolic events were observed. In a study injecting mesenchymal cells intra-arterially into the carotid artery, engraftment was observed without thromboembolic ischemia (9). Thromboembolic ischemia is measured with diffusion weighted imaging (DWI). This is an MRI technique that measures the microscopic movement of water. Using a DWI sequence, the signal intensity in ischemic brain regions is increased because the diffusion of coefficient of water is reduced, because of the formation of edema. This phenomenon can be quantified by the calculation of apparent diffusion coefficient (ADC) images. Following IA transplantation, a small cohort of experimental animals is run for a DWI sequence in order to determine if thromboembolic ischemia is present. With regard to possible concern relating to the timing of the transplantation with regards to the traumatic injury, one day was chosen because post-traumatic edema has not become a significant issue at this time (12).

### Survival of NPCs

NPC survival may be augmented by upregulating their expression of heat shock protein 70 (HSP70). Heat shock proteins are upregulated after brain injury due to ischemia or trauma, including in humans. As both caspase-mediated and AIF-mediated programmed cell death peak between 24-72 h after injury, when HSP70 proteins are upregulated by pharmacological agents in response to cellular stress, they provide strong cytoprotection against a large variety of insults. Thus, strategies to block stem cell death could have profound effects on outcome of the transplant.

### Methods

Neural progenitor cells are purchased from Lonza Corp (formerly Cambrex). They are exempt from embryonic stem cell guidelines. They grow as floating neurospheres in neural progenitor cell growth media containing EGF and bFGF. When neurospheres are dissociated and re-grown in neural progenitor cell medium containing 5% horse serum, the cells attach to the floor of culture flasks or plates. These attached cells consist of neuronal precursor cells, neurons, astrocytes, and oligodendrocytes. The attached neural progenitor cells are labeled with Feridex for 2 days before transplanted into host animals.

### Transfection

Mammalian expression plasmid bicistronic construct driving MT5-MMP and GFP are used. Stem cells are grown in vitro to moderate cell density and plasmid DNA transfected with the Lipofectamine transfection reagent for 4 hours prior to Feridex labeling.

Cell Labeling with Feridex

Feridex labeling solution was prepared by mixing Feridex I.V. (Ferumoxides injectable solution, 11.2 mg/ml; Berlex) and poly-l-lysine (Sigma, 1.5 mg/ml) in neural progenitor cell growth medium (final concentration: Feridex=50 ug/ml, poly-l-lysine=1 ug/ml).

### Feridex-Labeling of NPCs

Feridex and poly-l-lysine (33:1) in neural progenitor cell growth medium. Neural progenitor cells grown on flasks at 60-70% confluent are incubated with Feridex labeling solution for 2 days at 37° C., then dissociated for animal surgery and transplantation. Prussian blue stain is used to identify Feridex-labeled neural progenitor cells. Prussian blue staining solution consists of 1% Potassium Ferrocyanide and 1% Hydrochloric Acid. The staining were performed by 10 minutes incubation of Feridex-labeled neural progenitor cells with Prussian blue staining solution at room temperature.

### Placement of Magnet

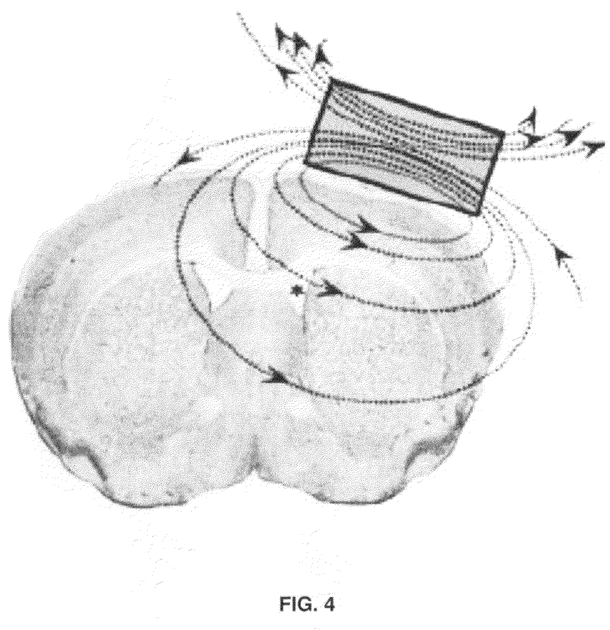

The magnet is placed on one side of the cortex (FIG. 4) over the impact site. Feridex-labeled stem cells are attracted by magnetic field gradient, which facilitate the retention of stem cells in the Injured ipsilateral cortex. The contralateral cortex is used as internal control. The distribution of GFP or Perl's labeled stem cells in the cortex is analyzed and the ratio of stem cells in both sides is calculated to confirm the target efficiency of stem cells on the side containing the magnet.

### CCI Model of TBI

Surgical anesthesia is induced by Nembutal (50 mg/kg). Analgesic buprenorphine is administered pre-emptively to alleviate the pain following surgery. A craniotomy is performed on the right hemisphere encompassing bregma and lambda and between the sagittal suture and the coronal ridge with a handheld Michele trephine. The resulting bone flap is removed. A cortical contusion is produced on the exposed cortex using a controlled impactor device TBI-0310 TBI Model system (Precision Systems and Instrumentation, LLC, Fairfax Station, Va.). Briefly, the impacting shaft is extended, and the impact tip is centered and lowered over the craniotomy site until it touches the dura mater. Then, the rod is retracted and the impact tip is advanced farther to produce a brain injury of moderate severity for rats (tip diameter, 4 mm; cortical contusion depth, 3 mm; impact velocity, 1.5 m/sec) (9). The impact tip is wiped clean with sterile alcohol after each impact and cleaned/disinfected further with cidex after surgery. Core temperature is maintained at 37±0.5° C. with a heating pad during surgery and recorded with a rectal probe. Immediately after impact followed by IVt injection, the skin incision is closed with nylon sutures, and 2% lidocaine jelly is applied to the lesion site to minimize any possible discomfort.

### Transplantation Surgery

Feridex labeled, MT5-MMP-GFP transfected neuroprogenitor stem cells are delivered into ventricles (lateral) in TBI rats or by IA injection. Ninety day old rats are anesthetized and labeled neuroprogenitor stem cells are injected into one of the lateral ventricle with a 0.6 T magnet placed on one side of the skull. Animals are euthanized by an overdose of Nembutal and the carotid and jugular veins are clamped and the brain dissected and placed in 4% PFA at 2 h, 1 d, or 7 d post-transplantation.

### Immunohistochemistry

Immunohistochemistry with GFP antibodies was used to analyze the tissue sections to identify the location of labeled neuroprogenitor stem cells. Meanwhile, the tissue sections labeled with Perl staining to label iron, which will confirm neuroprogenitor stem cells, are labeled by Feridex.

### Image Analysis

Sections are processed for Prussian blue staining to detect the presence of transplanted cells in the brain. Prussian blue-stained slices are selected to determine the number of neuroprogenitor stem cells that had migrated into the rat brain. GFP-immunostained neuroprogenitor stem cells are also counted. At least five fields of view (objective magnification, ×100) were selected randomly, photographed, and measured for Prussian blue staining, using an image analyzer program (Neurolucida). Similar to the previous distribution studies, Neurolucida is also used to plot position of labeled cells in cortical regions selected by systematic random stereological sampling both within the region of TBI and adjacent. The estimated number of cells in the regions, the apical-basal position, and estimated efficiency compared to the total number of cells injected are compared across the groups (multifactorial analysis of variance statistics).

### Statistical Analysis

All data in the present study are presented as means±SD. The nonparametric Mann-Whitney U test is used and p<0.05 is considered significant.

### Summary of Animal Numbers

(Intraventricular): 40 rats (10 rats for injection of Feridex-labeled neuroprogenitor stem cells into lateral ventricle under each of the 4 conditions following traumatic brain injury; For control non-traumatic brain injury rats, 20 rats (5 rats for injection of Feridex-labeled neuroprogenitor stem cells into lateral ventricle under each of the 4 conditions).

(Intra-arterial): 40 rats (10 rats for IA injection of Feridex-labeled neuroprogenitor stem cells under each of the 4 conditions following traumatic brain injury. For control non-traumatic brain injury rats, 20 rats (5 rats for injection of Feridex-labeled neuroprogenitor stem cells for IA injection under each of the 4 conditions).

The following references are cited herein:
1. Herr, H. J. of Rehab. Res. and Devel. 2002 39(3):11-12
2. Goodwin, S., J. of Magnetism and Magnetic Materials 1999 194:209-217
3. Liu et al. J. of Magnetism and Magnetic Materials 2001 225:209-217
4. Sheng et al. J. of Magnetism and Magnetic Materials 1999 194:167-175
5. Forbes et al. Abstract and Poster Presentation at the 6th Annual New Jersey Symposium on Biomaterials, Oct. 17-18, 2002, Somerset, N. J.
6. Johnson et al., Nat Rev Neurosci 2010; 11:361-370.
7. Harting et al., J Surg Res. 2009;153:188-194.
8. Heile et al., Neurosci Lett 2009; 463:176-181.
9. Lundberg et al., Neuroradiology 2009; 51:661-667.
10. Li et al., J Cereb Blood Flow Metab. 2010; 30:653-62.
11. Harting et al., J Neurosurg. 2009; 110:1189-1197.
12. Stoica B A and Faden A I. Neurotherapeutics. 2010; 7:3-12.
13. Fischer et al., Stem Cells Dev. 2009; 18:683-692.
14. Cernak et al., Neurobiol Dis. 2004; 17:29-43.
15. Fox et al., J Neurotrauma 1998; 15:599-614.
16. Khan et al., J Neuroinflammation. 2009; 6:32-44.
17. Xiong et al. Curr Opin Investig Drugs. 2010;11:298-308.
18. Parrish-Aungst et al. J. Comp Neurol, 2007, 501(6):825-836.
19. Kiyokage et al. J Neurosci, 2010, 30(3):1185-1196.

Any patents or publications mentioned in this specification are indicative of the levels of those skilled in the art to which the invention pertains. Further, these patents and publications are incorporated by reference herein to the same extent as if each individual publication was specifically and individually incorporated by reference. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A method of targeting stem cells to a specific region of the central nervous system of an individual in need of such treatment, comprising the steps of:
administering stem cells containing superparamagnetic nanoparticles to said individual; and
applying an external source of a magnetic field to the exterior of said individual's brain so as to advance and maneuver said stem cells.

2. The method of claim 1, wherein said stem cells are human neuroprogenitor stem cells.

3. The method of claim 1, wherein said superparamagnetic particle is selected from the group consisting of Feridex, Fe₂O₃, Fe₃O₄, and a superparamagnetic iron oxide nanoparticle.

4. The method of claim 1, wherein said individual has suffered a traumatic brain injury.

5. The method of claim 1, wherein said stem cells are administered by a route selected from the group consisting of intraventricular administration and intra-arterial injection.

6. The method of claim 1, wherein said stem cells contain a growth factor.

7. The method of claim 1, wherein said stem cells are transfected with a metalloproteinase.

8. The method of claim 7, wherein said metalloproteinase is MT5-MMP.

9. The method of claim 1, wherein said external source of a magnetic field is a fixed magnet having a strength between 0.3T and 6.0T.

10. The method of claim 1, wherein the amount of cells administered is from about 40,000/μl to about 80,000/μl.

11. The method of claim 1, wherein expression of heat shock protein 70 is upregulated in said stem cells.

12. The method of claim 1, wherein said individual has a disease or syndrome selected from the group consisting of Parkinson's Disease, Alzheimer's Disease, amyotrophic lateral sclerosis and traumatic brain injury.

13. The method of claim 1, wherein said individual has Parkinson's Disease and said cells are targeted to the striatum, said individual has Alzheimer's Disease and said cells are targeted to the cortex in or said individual has amyotrophic lateral sclerosis and said cells are targeted to the spinal cord.

14. A composition for targeting cells to a specific region of the central nervous system of an individual in need of such treatment, comprising:
stem cells containing superparamagnetic nanoparticles.

15. The composition of claim 14, wherein said stem cells are human neuroprogenitor stem cells.

16. The composition of claim 14, wherein said superparamagnetic particle is selected from the group consisting of Feridex, Fe₂O₃, Fe₃O₄ and a superparamagnetic iron oxide nanoparticle.

17. The composition of claim 14, wherein said stem cells contain a growth factor.

18. The composition of claim 14, wherein said stem cells are transfected with a metalloproteinase.

19. The composition of claim 18, wherein said metalloproteinase is MT5-MMP.

20. The composition of claim 14, wherein expression of heat shock protein 70 is upregulated in said stem cells.

21. A method of targeting superparamagnetic particle containing human neuroprogenitor stem cells to a specific region of the central nervous system of an individual in need of such treatment, comprising the steps of:
administering said stem cells to said individual; and
applying an external source of a magnetic field to the exterior of said individual's brain so as to advance and maneuver said stem cells.

22. The method of claim 21, wherein said superparamagnetic particle is selected from the group consisting of Feridex, Fe₂O₃, Fe₃O₄, and a superparamagnetic iron oxide nanoparticle.

23. The method of claim 21, wherein in said stem cells are administered by a route selected from the group consisting of intraventricular administration and intra-arterial injection.

24. The method of claim 21, wherein said stem cells contain a growth factor.

25. The method of claim 21, wherein said stem cells are transfected with a metalloproteinase.

26. The method of claim 25, wherein said metalloproteinase is MT5-MMP.

27. The method of claim 21, wherein said external source of a magnetic field is a fixed magnet having a strength between 0.3T and 6.0T.

28. The method of claim 21, wherein the amount of cells administered is from about 40,000/μl to about 80,000/μl.

29. The method of claim 21, wherein expression of heat shock protein 70 is upregulated in said stem cells.
